(19) United States
(12) Patent Application Publication
(10) Pub. No.: US 2003/0130244 A1
(43) Pub. Date: Jul. 10, 2003

(54) Compositions for treatment of postmenopausal female sexual dysfunction

(76) Inventors: Christopher R. Bilkey, Epping (AU); J. Greg Slatter, Chester, NJ (US); Ebrahim Versi, Gladstone, NJ (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/292,742

(22) Filed: Nov. 12, 2002

Related U.S. Application Data
(60) Provisional application No. 60/344,507, filed on Nov. 9, 2001.

(51) Int. Cl. A61K 31/56

(52) U.S. Cl. 514/170

(57) ABSTRACT

A set of pharmaceutical dosage forms is provided, each comprising at least two therapeutic agents selected from (a) an estrogen, (b) an androgen, and (c) an antimuscarinic, in total and relative dosage amounts that are therapeutically effective in treatment of female sexual dysfunction (FSD) or postmenopausal sexual avoidance (PMSA), said dosage forms being adapted for intravaginal administration. A method of treatment of FSD or PMSA comprises administering intravaginally, in a treatment regimen extending over a period of at least 7 days, dosage forms at least a portion of which comprise two or more therapeutic agents selected from (a) an estrogen, (b) an androgen, and (c) an antimuscarinic, in total and relative dosage amounts that are therapeutically effective in treatment of FSD or PMSA, wherein no more than one dosage form is administered on any day. Also provided is a kit useful in implementing such a treatment regimen.

This application claims priority of U.S. provisional application Serial No. 60/344,507 filed on Nov. 9, 2001.

### FIELD OF THE INVENTION

The present invention relates to pharmaceutical compositions in the form of vaginal dosage forms useful for treatment of symptoms related to female sexual dysfunction, and to therapeutic methods of use of such dosage forms.

### BACKGROUND OF THE INVENTION

Female sexual dysfunction (FSD) in postmenopausal women is a complex psychosexual disorder that can be treated hormonally with combinations of androgens and estrogens. See for example Berman et al. (1999), “Female sexual dysfunction: anatomy, physiology, evaluation and treatment options”, Curr. Opin. Urol. 9(6), 563-568; Berman et al. (2000), “Anatomy and physiology of female sexual function and dysfunction. Classification, evaluation and treatment options”, European Urology 38(1), 20-29; Berman et al. (2001), “Novel approaches to female sexual dysfunction”, Expert Opinion on Investigational Drugs 10(1), 85-95; Sarrel (1999), “Psychosexual effects of menopause: role of androgens”, Am. J. Obstet. Gynecol. 180(3 Part 2), S319-S324. An oral dosage form comprising esterified estrogens and the androgen methyltestosterone is commercially available, for example as Estratest® of Solvay Pharmaceuticals, and is disclosed by Berman et al. (1999), op. cit., to be useful in treatment of FSD. A topical testosterone cream, currently used in treating vulvar lichen planus, is indicated by Berman et al. (2000), op. cit., to have potential benefits including increased vaginal lubrication, increased libido and heightened arousal.

Classification of FSD is described by Basson et al. (2000), “Report of the international consensus development conference on female sexual dysfunction: definitions and classifications”, J. Urol. 163(3), 888-893. Guidance is available from the U.S. Federal Drug Administration (FDA) defining the disorder, subsets of women at risk, and appropriate study designs and clinical endpoints; see FDA Center for Drug Evaluation and Research (CDER) draft guidance document (May 2000) at www.fda.gov/cder/guidance/3312dft.htm.

The CDER draft guidance document referenced immediately above states that “the definition of FSD . . . currently consists of four recognized components: decreased sexual desire, decreased sexual arousal, dyspareunia, and persistent difficulty in achieving or inability to achieve orgasm. To establish a diagnosis of FSD, these components must be associated with personal distress, as determined by the affected woman.”

In a significant population of postmenopausal women, FSD as defined above is further compounded by atrophic vaginitis, a disorder characterized by vaginal dryness, soreness and/or irritation. Atrophic vaginitis makes sexual activity uncomfortable or painful, and is treatable with locally administered estrogen. For example, Vagifem® is a vaginal tablet containing 25 μg estradiol in a modified release matrix, indicated for post-menopausal atrophic vaginitis, and currently marketed by Pharmacia Corp. in North America. The Vagifem® tablet comprises a core having estradiol, in the form of estradiol hemihydrate, in a matrix comprising hydroxypropylmethylcellulose (HPMC), lactose monohydrate, corn starch and magnesium stearate, and a film coating comprising HPMC and polyethylene glycol (PEG). When the tablet is placed in the vagina, for example with the aid of a disposable applicator provided for this purpose, contact with the vaginal mucosa results in formation of a gel layer on the surface of the tablet. As moisture permeates the tablet, the tablet erodes and estradiol diffuses out of the gel layer into the mucosa. See Physicians' Desk Reference, 56th edition (2002), 2857-2860.

Libido in perimenopausal women has been found to be affected by anxiety and depression. See Channon & Ballinger (1986), “Some aspects of sexuality and vaginal symptoms during menopause and their relation to anxiety and depression”, Brit. J. Med. Psychol. 59, 173-180.

It has been noted that urinary incontinence can be associated with FSD in postmenopausal women. See Greendale et al. (2001), “Factors related to sexual function in postmenopausal women with a history of breast cancer”, Menopause 8(2), 111-119; and Lalos et al. (2001), “Impact of urinary and climacteric symptoms on social and sexual life after surgical treatment of stress urinary incontinence in women: a long-term outcome”, J. Adv. Nurs. 33(3), 316-327. Topical or intravaginal application of an estrogen such as estradiol is known to have a therapeutic effect in some cases of urinary incontinence, as disclosed or suggested by Batra & Iosif (1983), “Female urethra: a target for estrogen action”, J. Urol. 129(2), 418-420; Karram et al. (1989), “Management of coexistent stress and urge urinary incontinence”, Obstetrics & Gynecology 73(1); Goode et al. (1997), “Pharmacologic treatment of lower urinary tract dysfunction in geriatric patients”, Am. J. Med. Sci. 314(4), 262-267; and Lose & Englev (2000), “Oestradiol-releasing vaginal ring versus oestriol vaginal pessaries in the treatment of bothersome lower urinary tract symptoms”, Brit. J. Obst. Gyn. 107(8), 1029-1034.

U.S. Pat. No. 6,262,115 to Guittard et al. discloses oral administration of the antimuscarinic drug oxybutynin and estrogen in management of incontinence and hormone replacement therapy.

Olsson & Landgren (2001), “The effect of tolterodine on the pharmacokinetics and pharmacodynamics of a combination oral contraceptive containing ethinyl estradiol and levonorgestrel”, Clin. Therap. 23(11), 1876-1888, discloses oral administration of the antimuscarinic tolterodine in combination with an estrogen-containing oral contraceptive.

Some postmenopausal women have reported that urinary incontinence causes them to abstain from sexual activity, for example from fear of loss of urine during intercourse. However, a large number of women in all ethnic groups may be hesitant to seek medical help for chronic symptoms such as incontinence that affect intimate relationships, partly because discussing such matters is embarrassing for many women. See Barlow et al. (1997), “Urogenital aging and its effect on sexual health in older British women”, Brit. J. Obst. Gyn. 104(1), 87-91.

Reduced sexual activity from any cause can promote vaginal atrophy (see Leiblum et al., “Vaginal atrophy in the postmenopausal woman”, JAMA 249(16), 2195-2198) and can thereby lead to a self-perpetuating cycle that further compounds the problem of postmenopausal FSD.

The disclosure of all of the above referenced documents is incorporated herein by reference.

Postmenopausal FSD is therefore an under-recognized and under-treated disorder. Although therapies exist for individual disorders such as atrophic vaginitis (for example intravaginal estrogen, e.g., estradiol, administration), decreased sexual desire or arousal (for example androgen, e.g., testosterone, therapy) and urinary incontinence (for example oral administration of an antimuscarinic drug such as tolterodine) that can contribute to FSD, a significant unmet medical need remains for a treatment regimen that addresses a combination of two or more of such individual disorders, and for pharmaceutical compositions tailored to such a regimen.

### SUMMARY OF THE INVENTION

There is now provided a pharmaceutical dosage form comprising at least two agents selected from (a) an estrogen, (b) an androgen, and (c) an antimuscarinic, in total and relative dosage amounts that are therapeutically effective in treatment of FSD or disorders contributing thereto, said dosage form being adapted for intravaginal administration.

In one embodiment a vaginal dosage form of the invention comprises an estrogen and an androgen in total and relative dosage amounts that are therapeutically effective in treatment of FSD characterized at least by atrophic vaginitis and low libido.

In another embodiment a vaginal dosage form of the invention comprises an estrogen and an antimuscarinic in total and relative dosage amounts that are therapeutically effective in treatment of FSD characterized at least by atrophic vaginitis and anxiety arising from urinary incontinence.

In yet another embodiment a vaginal dosage form of the invention comprises an androgen and an antimuscarinic in total and relative dosage amounts that are therapeutically effective in treatment of FSD characterized at least by low libido and anxiety arising from urinary incontinence.

In yet another embodiment a vaginal dosage form of the invention comprises an estrogen, an androgen and an antimuscarinic in total and relative dosage amounts that are therapeutically effective in treatment of FSD characterized by atrophic vaginitis, low libido and anxiety arising from urinary incontinence.

There is further provided a method of treatment of FSD comprising administering intravaginally, in a treatment regimen extending over a period of at least 7 days, one to a plurality of dosage forms independently comprising one or more agents selected from (a) an estrogen, (b) an androgen, and (c) an antimuscarinic, wherein no more than one dosage form is administered on any day, wherein at least one such dosage form comprises at least two of said agents, and wherein said agents are administered in total and relative dosage amounts that are therapeutically effective in treatment of FSD or disorders contributing thereto.

There is still further provided a kit comprising a package having contained therein a plurality of vaginal dosage forms independently comprising one or more agents selected from (a) an estrogen, (b) an androgen and (c) an antimuscarinic, wherein at least one such dosage form comprises at least two of said agents, said package and/or said dosage forms bearing indicia identifying a day on which each dosage form is to be intravaginally administered, said indicia corresponding to a treatment regimen extending over a period of at least 7 days wherein no more than one dosage form is administered on any day, said treatment regimen providing administration of said agents in total and relative dosage amounts that are therapeutically effective in treatment of FSD or disorders contributing thereto.

A syndrome named “postmenopausal sexual avoidance” or PMSA is recognized herein. PMSA is characterized by the presence of any two of the following three contributory factors:

1. vaginal dryness, soreness or irritation of a severity sufficient to cause pain or discomfort during sexual intercourse, whether or not such vaginal dryness, soreness or irritation falls within a clinical definition of atrophic vaginitis, and whether or not such pain or discomfort falls within a clinical definition of dyspareunia;

2. low libido, including decreased sexual desire and/or arousal, associated with low testosterone level;

3. anxiety arising from urinary incontinence, such anxiety (a) being of a degree sufficient to cause the sufferer to refrain repeatedly, though not necessarily to totally abstain, from sexual intercourse, and (b) ranging from mere embarrassment to severe neurosis.

It will be noted that PMSA as defined above can, but does not necessarily, fall within an accepted clinical definition of FSD. Accordingly the following are embraced by the present invention:

a pharmaceutical dosage form comprising at least two agents selected from (a) an estrogen, (b) an androgen, and (c) an antimuscarinic, in total and relative dosage amounts that are therapeutically effective in treatment of PMSA, said dosage form being adapted for intravaginal administration;

a vaginal dosage form comprising an estrogen and an androgen in total and relative dosage amounts that are therapeutically effective in treatment of PMSA;

a vaginal dosage form comprising an estrogen and an antimuscarinic in total and relative dosage amounts that are therapeutically effective in treatment of PMSA;

a vaginal dosage form comprising an androgen and an antimuscarinic in total and relative dosage amounts that are therapeutically effective in treatment of PMSA;

a vaginal dosage form comprising an estrogen, an androgen and an antimuscarinic in total and relative dosage amounts that are therapeutically effective in treatment of PMSA;

a method of treatment of PMSA comprising administering intravaginally, in a treatment regimen extending over a period of at least 7 days, one to a plurality of dosage forms independently comprising one or more agents selected from (a) an estrogen, (b) an androgen, and (c) an antimuscarinic, wherein no more than one dosage form is administered on any day, wherein at least one such dosage form comprises at least two of said agents, and wherein said agents are administered in total and relative dosage amounts that are therapeutically effective in treatment of PMSA; and

a kit comprising a package having contained therein a plurality of vaginal dosage forms independently comprising one or more agents selected from (a) an estrogen, (b) an androgen and (c) an antimuscarinic, wherein at least one such dosage form comprises at least two of said agents, said package and/or said dosage forms bearing indicia identifying a day on which each dosage form is to be intravaginally administered, said indicia corresponding to a treatment regimen extending over a period of at least 7 days wherein no more than one dosage form is administered on any day, said treatment regimen providing administration of said agents in total and relative dosage amounts that are therapeutically effective in treatment of PMSA.

### BRIEF DESCRIPTION OF THE DRAWINGS

FIG. 1 is a diagram showing a PMSA treatment paradigm within which the present invention can be practiced.

FIG. 2 is a diagrammatic representation of a first kit of the invention, being a pack of vaginal tablets corresponding to a “cyclic” treatment regimen as described hereinbelow.

FIG. 3 is a diagrammatic representation of a second kit of the invention, being a pack of vaginal tablets corresponding to a “weekend” treatment regimen as described hereinbelow.

FIG. 4 is a diagrammatic representation of a third kit of the invention, being a pack of disposable vaginal applicators corresponding to a “weekend” treatment regimen as described hereinbelow.

### DETAILED DESCRIPTION OF THE INVENTION

The term “postmenopausal” herein relates to all stages of a woman's life from the onset of menopause onward, whether menopause occurs normally, prematurely or artificially, for example as a result of surgery. It is contemplated that certain benefits of the present invention will be afforded to perimenopausal or even to premenopausal women and the scope of the invention therefore extends to therapy for FSD or disorders contributory thereto at any stage of a woman's adult life. However, in preferred embodiments the invention is directed to postmenopausal FSD and in particular to PMSA as defined above.

The invention provides a novel and unique integrated approach, and pharmaceutical products useful therein, to treatment of PMSA. A paradigm for this approach is shown in FIG. 1, wherein the three contributory factors listed above are represented as interlocking circles. “Atrophic vaginitis” will be understood in FIG. 1 as an abbreviation for vaginal dryness, soreness or irritation of a severity sufficient to cause pain or discomfort during sexual intercourse, whether or not such vaginal dryness, soreness or irritation falls within a clinical definition of atrophic vaginitis, and whether or not such pain or discomfort falls within a clinical definition of dyspareunia. “Low libido” will be understood in FIG. 1 as an abbreviation for low libido, including decreased sexual desire and/or arousal, associated with low testosterone level. “Incontinence” will be understood in FIG. 1 as an abbreviation for anxiety arising from urinary incontinence, such anxiety (a) being of a degree sufficient to cause the sufferer to refrain repeatedly, though not necessarily to totally abstain, from sexual intercourse, and (b) ranging from mere embarrassment to severe neurosis.

PMSA is a syndrome characterized by at least two of these three factors; thus the areas of FIG. 1 where two or three circles overlap represent situations where the present invention has particular utility.

Area “EA” represents PMSA characterized by atrophic vaginitis and low libido, for which a vaginal dosage form of the invention comprising an estrogen and an androgen will be the treatment of choice.

Area “TE” represents PMSA characterized by incontinence and atrophic vaginitis, for which a vaginal dosage form of the invention comprising an antimuscarinic (e.g., tolterodine) and an estrogen will be the treatment of choice.

Area “TA” represents PMSA characterized by incontinence and low libido, for which a vaginal dosage form of the invention comprising an antimuscarinic (e.g., tolterodine) and an androgen will be the treatment of choice. This can also be the treatment of choice for women having all three contributory factors to PMSA, but who are already on an oral estrogen regimen, for example as hormone replacement therapy, or for whom estrogens are contraindicated for any reason.

Finally, area “TEA” represents PMSA characterized by all three contributory factors, for which a vaginal dosage form of the invention comprising an antimuscarinic (e.g., tolterodine), an estrogen and an androgen will be the treatment of choice, except as indicated immediately above.

Any dosage form adapted for intravaginal administration can be used according to the invention, including without limitation tablets, ovules, pessaries, creams, ointments, gels, foams, sponges and implants, preferably discrete unit dosage forms such as tablets, ovules and pessaries. Presently preferred dosage forms are vaginal tablets adapted for mucosal delivery of the therapeutic agents. Especially preferred are tablets that produce a gel layer on contact with the vaginal mucosa and that erode gradually to release the therapeutic agents for diffusion through the gel layer into the mucosa. Such tablets can illustratively be formulated similarly to Vagifem® vaginal tablets of Pharmacia Corp., but having at least two therapeutic agents as required herein as opposed to estradiol alone. Applicators, for example disposable applicators similar to those in which Vagifem® tablets are commercially supplied, can optionally be provided to facilitate intravaginal administration of tablets of the invention.

Accordingly in one series of embodiments there are provided:

a vaginal tablet comprising at least two agents selected from (a) an estrogen, (b) an androgen, and (c) an antimuscarinic, in total and relative dosage amounts that are therapeutically effective in treatment of FSD, disorders contributing thereto, or PMSA;

a vaginal tablet comprising an estrogen and an androgen in total and relative dosage amounts that are therapeutically effective in treatment of FSD, disorders contributing thereto, or PMSA;

a vaginal tablet comprising an estrogen and an antimuscarinic in total and relative dosage amounts that are therapeutically effective in treatment of FSD, disorders contributing thereto, or PMSA;

a vaginal tablet comprising an androgen and an antimuscarinic in total and relative dosage amounts that are therapeutically effective in treatment of FSD, disorders contributing thereto, or PMSA;

a vaginal tablet comprising an estrogen, an androgen and an antimuscarinic in total and relative dosage amounts that are therapeutically effective in treatment of FSD, disorders contributing thereto, or PMSA;

a vaginal tablet comprising an androgen in a dosage amount that, as part of a treatment regimen comprising co-therapy of the androgen with an estrogen and/or an antimuscarinic, is therapeutically effective in treatment of FSD, disorders contributing thereto, or PMSA;

a vaginal tablet comprising an antimuscarinic in a dosage amount that, as part of a treatment regimen comprising co-therapy of the antimuscarinic with an estrogen and/or an androgen, is therapeutically effective in treatment of FSD, disorders contributing thereto, or PMSA;

a method of treatment of FSD, disorders contributing thereto, or PMSA, the method comprising administering intravaginally, in a treatment regimen extending over a period of at least 7 days, one to a plurality of vaginal tablets independently comprising one or more agents selected from (a) an estrogen, (b) an androgen, and (c) an antimuscarinic, wherein no more than one tablet is administered on any day, wherein at least one such tablet comprises at least an androgen or an antimuscarinic, and wherein said agents are administered in total and relative dosage amounts that are therapeutically effective in treatment of FSD, disorders contributing thereto, or PMSA; and

a kit comprising a package having contained therein a plurality of vaginal tablets independently comprising one or more agents selected from (a) an estrogen, (b) an androgen and (c) an antimuscarinic, wherein at least one such tablet comprises at least an androgen or an antimuscarinic, said package and/or said tablets bearing indicia identifying a day on which each tablet is to be intravaginally administered, said indicia corresponding to a treatment regimen extending over a period of at least 7 days wherein no more than one tablet is administered on any day, said treatment regimen providing administration of said agents in total and relative dosage amounts that are therapeutically effective in treatment of FSD, disorders contributing thereto, or PMSA.

An embodiment of the invention is a vaginal dosage form, for example a vaginal tablet, comprising an androgen and at least one agent selected from an estrogen and an antimuscarinic, in total and relative dosage amounts that are therapeutically effective in treatment of FSD, disorders contributing thereto, or PMSA.

Therapeutic agents suitable as an estrogen component of a vaginal dosage form of the invention include nonsteroidal and steroidal types. Illustrative nonsteroidal estrogens include broparoestrol, chlorotrianisine, dienestrol, diethylstilbestrol, fosfestrol, hexestrol, methestrol and salts and esters thereof. Steroidal estrogens are presently preferred. Illustrative steroidal estrogens include colpormon, conjugated estrogenic hormones, equilenin, equilin, estradiol, estriol, estrone, ethinyl estradiol, mestranol, moxestrol, quinestradiol, quinestrol and salts and esters thereof. Estradiol is an especially preferred estrogen.

Therapeutic agents suitable as an androgen component of a vaginal dosage form of the invention illustratively include boldenone, cloxotestosterone, fluoxymesterone, mesterolone, methandrostenolone, methyltestosterone, norethandrolone, normethandrolone, oxandrolone, oxymesterone, oxymetholone, prasterone, stanolone, stanozolol, testosterone and salts and esters thereof. Methyltestosterone and testosterone are presently preferred.

Therapeutic agents suitable as an antimuscarinic component of a vaginal dosage form of the invention can be selected from antimuscarinics known to be effective for treating urinary incontinence or overactive bladder. Presently preferred antimuscarinics include oxybutynin, tolterodine and salts and esters thereof, more especially tolterodine and its pharmaceutically acceptable salts, for example tolterodine tartrate. Either the racemate or the S-enantiomer of tolterodine can be used. Alternatively, the 5-hydroxymethyl metabolite of tolterodine disclosed in International Patent Publication No. WO 94/11337, incorporated herein by reference, or its salts or esters can be used.

An appropriate dosage amount for each therapeutic agent can be selected based on readily available literature showing therapeutically effective doses of individual estrogens, androgens and antimuscarinics. In selecting suitable dosage amounts, it will be recognized that the objective of intravaginal estrogen administration is primarily local delivery, but that for the androgen and the antimuscarinic systemic delivery will generally be desired. It will further be recognized that delivery via the vaginal mucosa circumvents first-pass metabolism, thus dosage amounts lower than those typically administered orally may be effective.

In general, estradiol can be administered intravaginally in a dosage amount of about 10 to about 50 μg, preferably about 15 to about 40 μg, for example about 25 μg, no more than once daily. Other estrogens can be administered in dosage amounts therapeutically equivalent to these dosage amounts of estradiol.

A suitable dosage amount of methyltestosterone is likely to be found in the range of about 0.5 to about 2.5 mg, no more than once daily, but greater or lesser amounts can be safe and effective in particular cases. Other androgens can be administered in dosage amounts therapeutically equivalent to these dosage amounts of methyltestosterone. It will be found desirable to minimize the dosage amount of androgen and/or to minimize the number of days on which it is administered, to reduce risk of undesirable hepatic side-effects and masculinization.

A suitable amount of tolterodine is likely to be found in the range of about 0.1 to about 12 mg, preferably about 0.2 to about 6 mg, more preferably about 0.5 to about 5 mg, for example about 1 to about 2 mg, no more than once daily. Other antimuscarinics can be administered in dosage amounts therapeutically equivalent to these dosage amounts of tolterodine.

Dosage forms of the invention comprising tolterodine are preferably formulated to exhibit release characteristics providing a tolterodine pharmacokinetic profile by intravaginal administration appropriate for once-a-day or less frequent treatment. Release characteristics with respect to estrogen and/or androgen can also be consistent with once-a-day or less frequent administration.

A treatment regimen of the invention is implemented over a period described herein as a treatment cycle. Any convenient treatment cycle period of 7 days or longer can be used. A treatment cycle period of 28 days is often particularly convenient. A dosage form of the invention is administered intravaginally not less than once per treatment cycle, and not more than once per day during the treatment cycle.

In a typical treatment regimen, a vaginal dosage form comprising two or three therapeutic agents, i.e., a combination dosage form, is administered intermittently. On days when a combination dosage form is not administered, options include:

1. administration of a monotherapeutic dosage form, for example an estradiol tablet or a tolterodine tablet;

2. administration of a placebo dosage form, i.e., a dosage form containing no therapeutic agent; or

3. no administration.

An advantage of the present invention is that the treatment regimen can be tailored precisely to the particular symptoms exhibited by the patient. A further advantage is that the treatment regimen can be designed to provide maximum relief of symptoms, and thereby stimulate interest in sexual activity, at convenient or predictable times, such on a 28-day cycle or at weekends.

Illustratively, a “cyclic” treatment regimen lasting 28 days, for a woman receiving estrogen and androgen combination therapy, could be as shown below:

| | Mon | Tue | Wed | Thu | Fri | Sat | Sun |
|---|---|---|---|---|---|---|---|
| Week 1 | E | x | x | x | B | x | x |
| Week 2 | EA | A | A | A | EA | A | A |
| Week 3 | B | x | x | x | B | x | x |
| Week 4 | B | x | x | x | B | x | x |

where x represents either placebo or no administration, E represents estrogen monotherapy, A represents androgen monotherapy and EA represents administration of an estrogen+androgen dosage form in accordance with the invention. For a woman additionally requiring tolterodine for treatment of an incontinence component of PMSA, an illustrative “cyclic” treatment regimen could be:

| | Mon | Tue | Wed | Thu | Fri | Sat | Sun |
|---|---|---|---|---|---|---|---|
| Week 1 | TE | T | T | T | TE | T | T |
| Week 2 | TEA | TA | TA | TA | TEA | TA | TA |
| Week 3 | TE | T | T | T | TE | T | T |
| Week 4 | TE | T | T | T | TE | T | T |

where T represents tolterodine monotherapy, TE represents administration of a tolterodine+estrogen dosage form, TA represents administration of a tolterodine+androgen dosage form, and TEA represents administration of a tolterodine+estrogen+androgen dosage form in accordance with the invention.

Illustratively, a “weekend” treatment regimen lasting 28 days, for a woman receiving tolterodine, estrogen and androgen combination therapy, could be as shown below:

| | Mon | Tue | Wed | Thu | Fri | Sat | Sun |
|---|---|---|---|---|---|---|---|
| Week 1 | TE | T or x | T or x | T or x | TE | TA | TA |
| Week 2 | TE | T or x | T or x | T or x | TE | TA | TA |
| Week 3 | TE | T or x | T or x | T or x | TE | TA | TA |
| Week 4 | TE | T or x | T or x | T or x | TE | TA | TA |

where x, T, TB and TA are as defined above.

A treatment regimen requiring administration of different tablets on different days can be inconvenient for the patient and can result in poor compliance. To avoid this problem, it is contemplated that tablets can be packaged in “kit” form, with labeling or other indicia on the package and/or on the tablets themselves to ensure the patient administers the correct tablet on each day. Accordingly, an embodiment of the present invention is a kit comprising a package having contained therein a plurality of vaginal tablets, at least a portion of which comprise two or more of estrogen, androgen and tolterodine, said package and/or said tablets bearing indicia identifying a day on which each dosage form is to be administered. If a separate disposable applicator is provided for each tablet as a component of the package, the indicia can be on the applicators.

The indicia can be numerical, e.g., 1, 2, 3, etc.; representative of days of the week, e.g., M, T, W, etc.; or otherwise indicative of a particular day in the treatment cycle. Optionally, the tablets and/or disposable vaginal applicators therefor can in addition be color coded or otherwise visually differentiated.

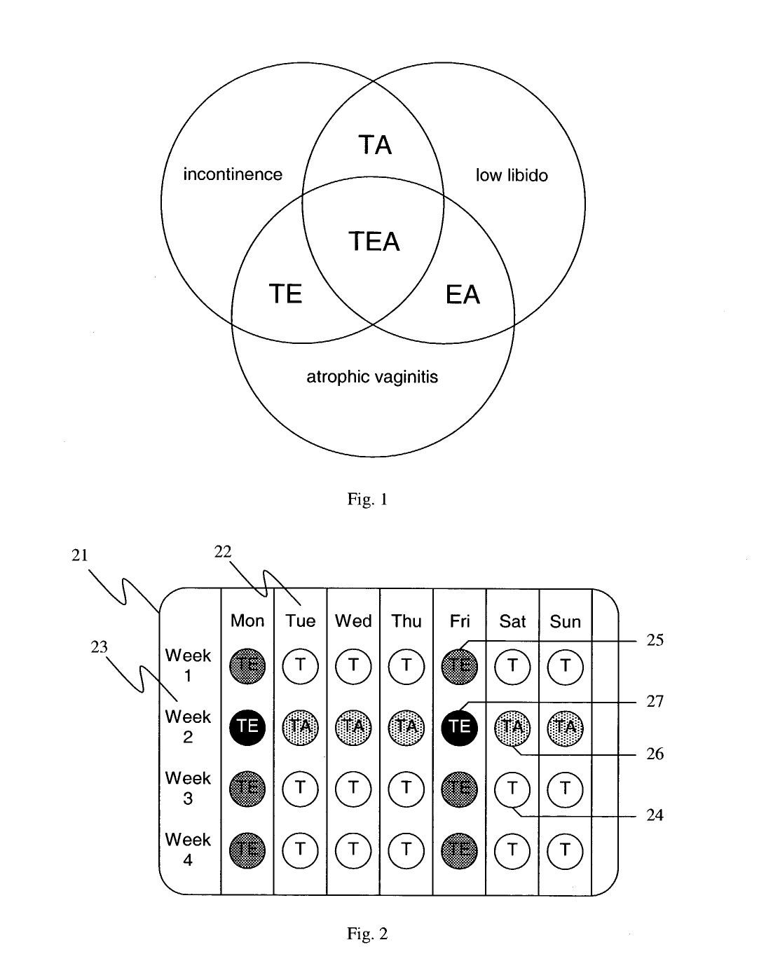

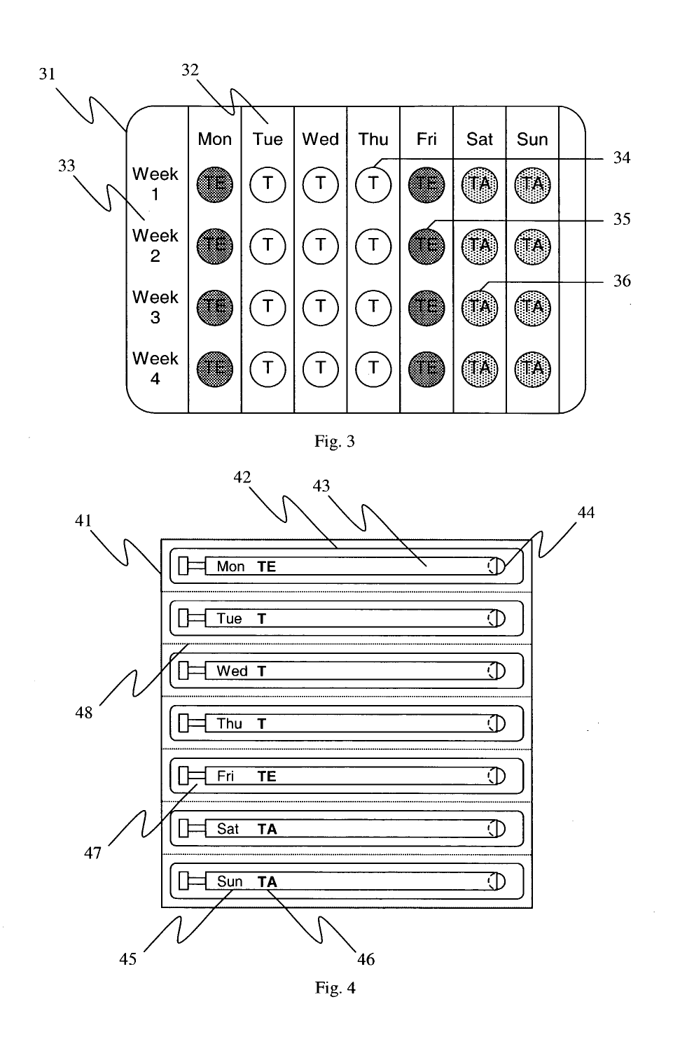

Any suitable package configuration can be employed, for example a rectangular matrix as illustrated in FIGS. 2 and 3, or a “dial-a-dose” package as is sometimes used for oral contraceptives.

FIG. 2 shows an illustrative kit in the form of a rectangular blister package of tablets suitable for a “cyclic” treatment regimen involving tolterodine, estrogen and androgen. A blister pack 21 is marked with day indicia 22, in this case representative of days of the week Monday through Sunday, and week indicia 23, in this case numerical. The blister pack holds twenty-eight vaginal tablets, some of which 24 contain tolterodine as sole therapeutic agent, others 25 contain tolterodine+estrogen, still others 26 contain tolterodine+androgen, and yet others 27 contain tolterodine+estrogen+androgen. Also provided as part of the kit (not shown) is a product label insert providing directions for use and other necessary information, and optionally one or more vaginal applicators.

FIG. 3 shows a kit in the form of a rectangular blister package of tablets suitable for a “weekend” treatment regimen involving tolterodine, estrogen and androgen. A blister pack 31 is marked with day indicia 32, in this case representative of days of the week Monday through Sunday, and week indicia 33, in this case numerical. The blister pack holds twenty-eight vaginal tablets, some of which 34 contain tolterodine as sole therapeutic agent, others 35 contain tolterodine+estrogen, and still others 36 contain tolterodine+androgen. Also provided as part of the kit (not shown) is a product label insert providing directions for use and other necessary information, and optionally one or more vaginal applicators.

FIG. 4 shows a seven-day kit suitable for a “weekend” treatment regimen. A package 41 comprises seven independently sealed but openable compartments 42 each containing a disposable vaginal applicator 43 having a vaginal tablet 44 dischargeably disposed therein. The compartments 42 and/or applicators 43 are marked with day indicia 45, in this case representative of days of the week Monday through Sunday, and/or indicia 46 representative of the therapeutic agent or agents present in the tablet disposed in each applicator, in this case T for tolterodine, TA for tolterodine+androgen, and TE for tolterodine+estrogen. Preferably the package 41 has a transparent wall 47 and the day indicia 45 and/or therapeutic agent indicia 46 are on the applicators 43 and are legible through the wall 47. Optionally the package 41 has lines of weakness, for example, perforations 48, between compartments 42 to facilitate tearing of the package into single-compartment pieces without unsealing the compartments.

The packages illustrated in FIGS. 2, 3 and 4 are preferably enclosed within an outer package (not shown).

### EXAMPLES

The following examples illustrate aspects of the present invention but are not to be construed as limitations.

### Example 1

A vaginal tablet containing 25 μg estradiol and 1 mg methyltestosterone is formulated similarly to Vagifem® tablets except for the addition of the methyltestosterone.

The tablet is useful as part of a treatment regimen for PMSA. The estradiol is delivered primarily locally for relief of vaginal dryness, soreness and/or irritation. The methyltestosterone is delivered systemically to increase libido.

### Example 2

A vaginal tablet containing 25 μg estradiol and 2 mg tolterodine tartrate is formulated similarly to Vagifem® tablets except for the addition of the tolterodine tartrate.

The tablet is useful as part of a treatment regimen for PMSA. The estradiol is delivered primarily locally for relief of vaginal dryness, soreness and/or irritation. The tolterodine is delivered systemically to control urinary incontinence and thereby remove a source of anxiety contributing to PMSA.

### Example 3

A vaginal tablet containing 1 mg methyltestosterone and 2 mg tolterodine tartrate is formulated similarly to Vagifem® tablets except for replacement of estradiol by methyltestosterone and tolterodine tartrate.

The tablet is useful as part of a treatment regimen for PMSA. The methyltestosterone is delivered systemically to increase libido. The tolterodine is delivered systemically to control urinary incontinence and thereby remove a source of anxiety contributing to PMSA.

### Example 4

A vaginal tablet containing 25 μg estradiol, 1 mg methyltestosterone and 2 mg tolterodine tartrate is formulated similarly to Vagifem® tablets except for the addition of the methyltestosterone and the tolterodine tartrate.

The tablet is useful as part of a treatment regimen for PMSA. The estradiol is delivered primarily locally for relief of vaginal dryness, soreness and/or irritation. The methyltestosterone is delivered systemically to increase libido. The tolterodine is delivered systemically to control urinary incontinence and thereby remove a source of anxiety contributing to PMSA.

### Example 5

A vaginal tablet containing 1 mg methyltestosterone is formulated similarly to Vagifem® tablets except for replacement of estradiol by methyltestosterone.

The tablet is useful as part of a treatment regimen for PMSA. The methyltestosterone is delivered systemically to increase libido.

### Example 6

A vaginal tablet containing 2 mg tolterodine tartrate is formulated similarly to Vagifem® tablets except for replacement of estradiol by tolterodine tartrate.

The tablet is useful as part of a treatment regimen for PMSA. The tolterodine is delivered systemically to control urinary incontinence and thereby remove a source of anxiety contributing to PMSA.

What is claimed is:

1. A pharmaceutical dosage form comprising at least two agents selected from the group consisting of (a) an estrogen, (b) an androgen, and (c) an antimuscarinic, in total and relative dosage amounts that are therapeutically effective in treatment of female sexual dysfunction or disorders contributing thereto, said dosage forms being adapted for intravaginal administration.

2. The dosage form of claim 1 comprising an estrogen and an antimuscarinic in total and relative dosage amounts that are therapeutically effective in treatment of female sexual dysfunction characterized at least by atrophic vaginitis and anxiety arising from urinary incontinence.

3. The dosage form of claim 1 that is a vaginal dosage form selected from the group consisting of tablets, ovules, pessaries, creams, ointments, gels, foams, sponges and implants.

4. The dosage form of claim 1 that is a vaginal tablet.

5. The dosage form of claim 1 wherein the estrogen if present is a nonsteroidal estrogen selected from the group consisting of broparoestrol, chlorotrianisine, dienestrol, diethylstilbestrol, fosfestrol, hexestrol, methestrol and salts and esters thereof.

6. The dosage form of claim 1 wherein the estrogen if present is a steroidal estrogen selected from the group consisting of colpormon, conjugated estrogenic hormones, equilenin, equilin, estradiol, estriol, estrone, ethinyl estradiol, mestranol, moxestrol, quinestradiol, quinestrol and salts and esters thereof.

7. The dosage form of claim 1 wherein the estrogen if present is estradiol.

8. The dosage form of claim 1 wherein the androgen if present is selected from the group consisting of boldenone, cloxotestosterone, fluoxymesterone, mesterolone, methandrostenolone, methyltestosterone, norethandrolone, normethandrolone, oxandrolone, oxymesterone, oxymetholone, prasterone, stanolone, stanozolol, testosterone and salts and esters thereof.

9. The dosage form of claim 1 wherein the androgen if present is methyltestosterone.

10. The dosage form of claim 1 wherein the androgen if present is testosterone.

11. The dosage form of claim 1 wherein the antimuscarinic if present is selected from the group consisting of oxybutynin, tolterodine, the 5-hydroxymethyl metabolite of tolterodine and salts and esters thereof.

12. The dosage form of claim 1 wherein the antimuscarinic if present is tolterodine or a pharmaceutically acceptable salt thereof.

13. A pharmaceutical dosage form comprising at least two agents selected from the group consisting of (a) an estrogen, (b) an androgen, and (c) an antimuscarinic, in total and relative dosage amounts that are therapeutically effective in treatment of postmenopausal sexual avoidance, said dosage forms being adapted for intravaginal administration.

14. The dosage form of claim 13 comprising an estrogen and an antimuscarinic in total and relative dosage amounts that are therapeutically effective in treatment of postmenopausal sexual avoidance.

15. The dosage form of claim 13 that is a vaginal dosage form selected from the group consisting of tablets, ovules, pessaries, creams, ointments, gels, foams, sponges and implants.

16. The dosage form of claim 13 that is a vaginal tablet.

17. The dosage form of claim 13 wherein the estrogen if present is a nonsteroidal estrogen selected from the group consisting of broparoestrol, chlorotrianisine, dienestrol, diethylstilbestrol, fosfestrol, hexestrol, methestrol and salts and esters thereof.

18. The dosage form of claim 13 wherein the estrogen if present is a steroidal estrogen selected from the group consisting of colpormon, conjugated estrogenic hormones, equilenin, equilin, estradiol, estriol, estrone, ethinyl estradiol, mestranol, moxestrol, quinestradiol, quinestrol and salts and esters thereof.

19. The dosage form of claim 13 wherein the estrogen if present is estradiol.

20. The dosage form of claim 13 wherein the androgen if present is selected from the group consisting of boldenone, cloxotestosterone, fluoxymesterone, mesterolone, methandrostenolone, methyltestosterone, norethandrolone, normethandrolone, oxandrolone, oxymesterone, oxymetholone, prasterone, stanolone, stanozolol, testosterone and salts and esters thereof.

21. The dosage form of claim 13 wherein the androgen if present is methyltestosterone.

22. The dosage form of claim 13 wherein the androgen if present is testosterone.

23. The dosage form of claim 13 wherein the antimuscarinic if present is selected from the group consisting of oxybutynin, tolterodine, the 5-hydroxymethyl metabolite of tolterodine and salts and esters thereof.

24. The dosage form of claim 13 wherein the antimuscarinic if present is tolterodine or a pharmaceutically acceptable salt thereof.
